(19) United States
(12) Patent Application Publication
(10) Pub. No.: US 2015/0284305 A1
(43) Pub. Date: Oct. 8, 2015

(54) FLUORINE-CONTAINING COMPOUND AND METHOD FOR PRODUCING SAME

(71) Applicant: Asahi Glass Company, Limited, Tokyo (JP)

(72) Inventors: Tomoko YAJIMA, Tokyo (JP); Emiko NOGAMI, Tokyo (JP); Kyoko YAMAMOTO, Tokyo (JP)

(73) Assignee: Asahi Glass Company, Limited, Tokyo (JP)

(21) Appl. No.: 14/745,103

(22) Filed: Jun. 19, 2015

Related U.S. Application Data
(63) Continuation of application No. PCT/JP2013/083668, filed on Dec. 16, 2013.

(30) Foreign Application Priority Data
Dec. 21, 2012 (JP) 2012-280153

(51) Int. Cl. C07C 17/02; C07C 22/08

(52) U.S. Cl. CPC C07C 17/02; C07C 22/08

(57) ABSTRACT

The present invention relates to a method containing reacting a compound represented by the following formula (1) and a compound represented by a formula R^{f1}X under light irradiation in a halogen-containing solvent in the presence of a thiosulfate salt and thereby obtaining a fluorine-containing compound represented by the following formula (2) and/or a fluorine-containing compound represented by the following formula (3), in which X represents an iodine atom or a bromine atom, R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of 1 or more and 4 or less,

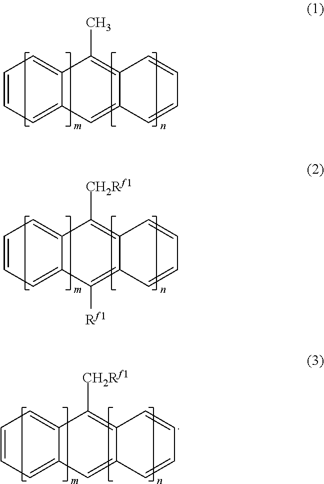

### TECHNICAL FIELD

The present invention relates to a novel fluorine-containing compound applicable to organic semiconductor materials, and to a method for producing the same.

Recently, an organic semiconductor element using an organic compound as the semiconductor material has an easy workability, as compared with an already-existing semiconductor element using an inorganic semiconductor material such as silicon or the like, and is therefore expected to realize low-cost devices. In addition, a semiconductor material of an organic compound is structurally flexible and is, as combined with a plastic substrate, therefore expected to realize flexible devices such as displays, etc.

As a semiconductor working process, there are known a dry process through vapor deposition with plasma, ion beams or the like and a wet process using an organic solvent, such as coating, printable, inkjet or the like process. Already-known organic semiconductor materials have low solubility in organic solvents and are therefore difficult to apply to a wet process, for which, therefore, a dry process has been widely utilized. On the other hand, a wet process has advantages such as workability not giving any damage to semiconductor crystals.

In general, an organic semiconductor material is required to have improved carrier mobility. Any effective means has not as yet been established for improving the carrier mobility of an organic semiconductor material, but it is considered important to strengthen the intermolecular interaction and to control the molecular arrangement. For example, since a conjugated system is expanded owing to the planar structure therein, a condensed polycyclic compound secures strong intermolecular interaction through π-π stacking and therefore use thereof as an organic semiconductor material is tried (Non-Patent Document 1).

An acene compound of a condensed polycyclic compound is expected to exhibit an excellent function as an organic semiconductor material. An acene compound is a compound having a skeleton of benzene rings linearly condensed to each other. Such an acene compound has a small theoretical band bap as compared with polyacetylene or the like, and is expected to have an excellent function as an organic semiconductor material. In addition, with increase in the number of the constituent rings, the compound is expected to exhibit a more excellent function.

However, the solubility of the acene compound in organic solvents decreases with the increase in the constituent rings. Accordingly, it is difficult to apply a wet process to an acene compound, and therefore the latitude in selecting solvent, temperature condition and others is extremely narrow.

Patent Document 1 discloses a method for increasing the solubility into an organic solvent by introducing a substituent such as an alkyl group or the like into the acene structure in order to use the acene compound as the organic semiconductor material by the wet process. Patent Document 2 discloses a method for producing an acene compound having a perfluoroalkyl group according to a coupling reaction using a heavy metal.

Patent Document 3 and Non-Patent Document 2 disclose perfluoroalkylation of benzenes for direct application of polyfluoroalkylation to aromatic compounds.

Non-Patent Document 3 discloses a naphthalene compound with a 2,2,2-trifluoroethyl group bonding thereto.

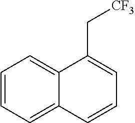 [SMILES: FC(F)(F)Cc1cccc2ccccc12]

### PRIOR ART DOCUMENT

### Patent Document

Patent Document 1: JP-A 2007-13097
Patent Document 2: WO 2011/022678
Patent Document 3: U.S. Pat. No. 3,271,441

### Non-Patent Document

Non-Patent Document 1: D. J. Gundlach, S. F. Nelson, T. N. Jachson, et al., Appl. Phys. Lett., (2002), 80, 2925.
Non-Patent Document 2: Anna Bravo, et al., J. Org. Chem., (1997), 62, 7128.
Non-Patent Document 3: Uneyama Kenji, et al., Tetrahedron Lett., (1989), 30, 2265.

### SUMMARY OF INVENTION

### Problem to be Solved by the Invention

However, Patent Document 1 does not disclose a compound having a fluorine-containing alkyl group. In addition, in the production method in Patent Document 2, a halo-substituted acene compound and a perfluoroalkyl iodide are subjected to coupling reaction in the presence of a heavy metal, and therefore the synthesis is complicated and there is another problem of contamination with the heavy metal. In general, an organic semiconductor material is required to have a high purity, and therefore such heavy metal contamination results in the necessity of a labor-intensive process of ultra-purification through sublimation purification or the like.

Patent Document 3 and Non-Patent Document 2 disclose perfluoroalkylation of benzenes not using heavy metal coupling reaction, but do not disclose introduction of a fluorine-containing alkyl group into acene compounds.

Non-Patent Document 3 does neither disclose nor suggest any other compound than the above-mentioned compound.

The first object of the present invention is to provide a compound applicable to both a dry process and a wet process and useful as an organic semiconductor material having a high carrier mobility.

The second object of the present invention is to provide a method for producing the compound that carries little risk of contamination with a heavy metal, which may be a cause of lowering the carrier mobility.

### Means for Solving the Problem

The present inventors have newly found out an acene-type fluorine-containing compound having a group —CH₂R^{f1} as a substituent therein, and simultaneously have found out a production method for the compound capable of evading the risk of contamination with a heavy metal by employing photoradical reaction.

That is, the present invention relates to the following 1 to 15.

1. A fluorine-containing compound represented by the following formula (2).

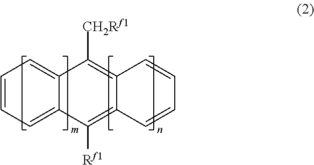

In the above formula, R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of 1 or more and 4 or less.

2. The fluorine-containing compound according to the above item 1, in which R^{f1} is a perfluoroalkyl group having a carbon number of from 1 to 12.
3. The fluorine-containing compound according to the above item 1, in which R^{f1} is a linear-structured perfluoroalkyl group having a carbon number of from 1 to 12, m is an integer of 1 or more, n is an integer of 1 or more, and m+n is an integer of 2 or more and 4 or less.
4. An organic semiconductor material containing the fluorine-containing compound described in any one of the above items 1 to 3.
5. An organic semiconductor thin film containing the fluorine-containing compound described in any one of the above items 1 to 3.
6. The organic semiconductor thin film according to the above item 5, in which the organic semiconductor thin film is a crystalline thin film.
7. An organic semiconductor element containing a layer of the organic semiconductor thin film described in the above item 5 or 6, as the semiconductor layer therein.
8. An organic semiconductor transistor containing the organic semiconductor element described in the above item 7.
9. A method for producing a fluorine-containing compound represented by the following formula (2), which containing reacting a compound represented by the following formula (1) and a compound represented by a formula R^{f1}X (in which X represents an iodine atom or a bromine atom) under irradiation with light in a halogen-containing solvent and in the presence of a thiosulfate salt.

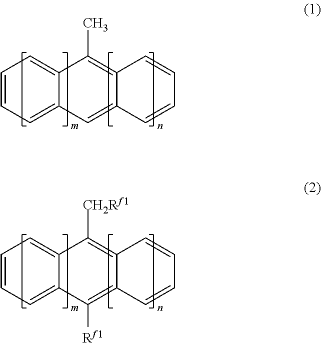

In the above formulae, R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of 1 or more and 4 or less.

10. The production method according to the above item 9, in which R^{f1} is a perfluoroalkyl group having a carbon number of from 1 to 12.
11. The production method according to the above item 9 or 10, in which R^{f1} is a linear-structured perfluoroalkyl group having a carbon number of from 1 to 12, m is an integer of 1 or more, n is an integer of 1 or more, and m+n is an integer of 2 or more and 4 or less.
12. A method for producing a fluorine-containing compound represented by the following formula (2) and a fluorine-containing compound represented by the following formula (3), which contains reacting a compound represented by the following formula (1) and a compound represented by a formula R^{f1}X (in which X represents an iodine atom or a bromine atom) under irradiation with light in a halogen-containing solvent and in the presence of a thiosulfate salt.

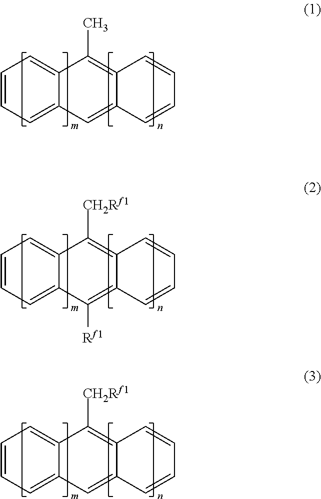

In the above formulae, R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of 1 or more and 4 or less.

13. The production method according to the above item 12, in which R^{f1} is a perfluoroalkyl group having a carbon number of from 1 to 12.
14. The production method according to the above item 12 or 13, in which R^{f1} is a linear-structured perfluoroalkyl group having a carbon number of from 1 to 12, m is an integer of 1 or more, n is an integer of 1 or more, and m+n is an integer of 2 or more and 4 or less.
15. A compound represented by the following formula (3′).

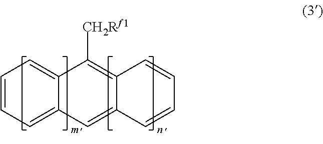

In the above formula, R^{f1} represents a fluorine-containing alkyl group having a carbon number of from 1 to 12, in which one or more fluorine atoms directly bond to the carbon atom that serves as an atomic bond, m′ is an integer of 1 or more, n′ is an integer of 0 or more, and m′+n′ is an integer of 2 or more and 4 or less.

### Advantageous Effects of Invention

In the fluorine-containing compound of the present invention, the conjugated system is expanded owing to the planar structure formed by the aromatic rings therein and the compound therefore has a strong intermolecular interaction through π-π stacking. Further, the compound has a skeleton of linearly-condensed aromatic rings and therefore the theoretical bandgap thereof is small as compared with that of other condensed compounds, and therefore the compound can exhibit an excellent function as an organic semiconductor material.

Moreover, introducing a fluorine-containing group having a specific structure into the compound facilitates the dissolution of the compound into a low-polar solvent. Accordingly, it becomes possible to produce an organic semiconductor material not only through a dry process but also through a wet process. In addition, the introduction of a fluorine-containing group having a specific structure enhances the intramolecular interaction utilizing the fluorophilic effect of the fluorine atom, and therefore the resultant compound can exhibit a higher carrier mobility as an organic semiconductor material.

According to the production method of the present invention, since a heavy metal catalyst is not used in the production step, it is possible to prevent contamination with the heavy metal that causes degradation in the carrier mobility of the produced compound, and therefore it is possible to provide a charge-transporting material having a high carrier mobility.

### MODES FOR CARRYING OUT THE INVENTION

The present invention is described in detail hereinunder, but the present invention is not limited to the embodiments described below and can be modified and changed in any desired manner within a scope not overstepping the scope and the spirit of the present invention.

In this description, a compound represented by a formula (X) may be referred to as “compound (X)”. The carrier mobility as referred to in this description is a comprehensive term that includes both an electron mobility and a hole mobility.

In addition, in this description, “% by mass” and “% by weight” are synonyms, and “mass ppm” and “weight ppm” are synonyms.

### [Fluorine-Containing Compound]

The present invention provides a novel fluorine-containing compound represented by the following formula (2). In addition, the present invention provides a novel compounds represented by the following formula (3), which is a reaction intermediate of the compound represented by the formula (2).

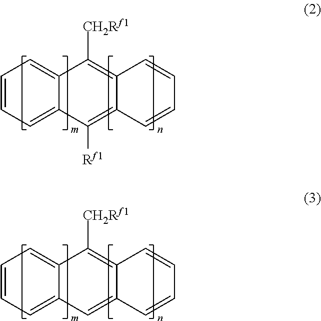

In the formula (2) and the formula (3), R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of from 1 or more and 4 or less.

In the formula (2) and the formula (3), R^{f1} may also be represented by the following formula (4).

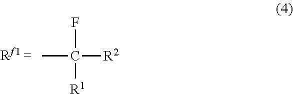

In the above formula (4), R¹ and R² each independently represent hydrogen atom, fluorine atom, or a fluorine-containing alkyl group having a carbon number of from 1 to 11, and the total carbon number of R¹ and R² is from 1 to 11.

The fluorine-containing alkyl group is a group derived from an alkyl group by replacing one or more constitutive hydrogen atoms with fluorine atom(s).

The condensed polycyclic compound is expected to have an improved carrier mobility owing to the strong intermolecular interaction through the π-π stacking of the condensed rings, but tends to have a low solubility in organic solvents.

With that, a part of the hydrogen atoms in the condensed polycyclic compound are replaced with a fluorine-containing alkyl group (R^{f1}) to increase the solubility of the compound in organic solvents. In addition, by increasing the intermolecular force through the fluorophilic effect of R^{f1}, a fluorine-containing compound excellent in oxidation resistance and having high sublimability is expected.

Further, by making the substituent bonding to the acene skeleton as —CH₂R^{f1}, not as —R^{f1}, the solubility of the compound in organic solvents can be further improved. It is also expected that the π-π stacking distance can be controlled, the HOMO-LUMO energy bandgap can be widened, and the light resistance of the compound can be further improved.

However, when the carbon number of the fluorine-containing alkyl group is too large, then the intermolecular interaction effect between the condensed rings would be weakened owing to steric hindrance, and therefore, the carbon number of the fluorine-containing alkyl group is preferably from 1 to 12. Above all, from the viewpoint of the balance between the intermolecular interaction and the improvement in solubility, the carbon number is more preferably from 2 to 10.

In the compound (2), the fluorine-containing alkyl group (R^{f1}) is preferably a perfluoroalkyl group from the viewpoint of the fluorophilic effect. The perfluoroalkyl group means an alkyl group in which all the constitutive hydrogen atoms are replaced with fluorine atoms. The perfluoroalkyl group is preferably a linear group represented by —(CF₂)ₖCF₃ (in which k is an integer of from 1 to 11). Concretely, the perfluoroalkyl group is preferably a perfluoroalkyl group having a carbon number of from 1 to 12. From the viewpoint of the balance between the intermolecular interaction and the improvement in solubility, a group having a carbon number of from 2 to 10 is more preferable, and a group in which the above-mentioned k is from 1 to 9 is even more preferable.

From the viewpoint of the solubility of the compound into organic solvents, R^{f1} is preferably a linear-structured (linear) group. Accordingly, more preferred is a linear perfluoroalkyl group having a carbon number of from 1 to 12, and even more preferred is a linear perfluoroalkyl group having a carbon number of from 2 to 10.

Concretely, from the viewpoint of the characteristics of the compound as an organic semiconductor and from the viewpoint of the production yield thereof, R^{f1} includes trifluoromethyl group, perfluoroethyl group, perfluoro-n-propyl group, perfluoro-isopropyl group, perfluoro-n-butyl group, perfluoro-isobutyl group, perfluoro-sec-butyl group, perfluoro-n-pentyl group, perfluoro-n-hexyl group, perfluoro-n-heptyl group, and perfluoro-n-octyl group.

From the viewpoint of the solubility in organic solvents, R^{f1} is preferably a linear-structured substituent, and is especially preferably trifluoromethyl group, perfluoroethyl group, perfluoro-n-propyl group, perfluoro-n-butyl group, perfluoro-n-hexyl group, perfluoro-n-heptyl group, or perfluoro-n-octyl group.

In the compound (2), m and n each indicate the repeating number of the structural units of a benzene ring, and it is preferred that m is an integer of 1 or more, n is an integer of 0 or more and m+n is an integer of 1 or more and 4 or less. n of 0 means the absence of the benzene ring surrounded by [ ] (or that is, the compound (2) is a condensed ring compound having (m+1)'s ring structures).

Preferably, m is an integer of 1 or more, n is an integer of 1 or more, and m+n is an integer of 2 or more and 4 or less. Of the case, from the viewpoint of the solubility in organic solvents, m is more preferably 1 or 2 and n is more preferably 1 or 2.

R^{f1} in the compound (3) is preferably a perfluoroalkyl group from the viewpoint of the fluorophilic effect, like the above-mentioned R^{f1} in the compound (2). From the viewpoint of the solubility in organic solvents, preferred is a linear-structured group.

Concretely, preferred is a perfluoroalkyl group having a carbon number of from 1 to 12, and more preferred is a perfluoroalkyl group having a carbon number of from 2 to 10. Also preferred is a linear-structured perfluoroalkyl group having a carbon number of from 1 to 12, and especially preferred is a linear-structured perfluoroalkyl group having a carbon number of from 2 to 10.

More concretely, preferably used here are the same substituents as those exemplified for the previously-mentioned R^{f1} in the compound (2).

In the compound (3), m and n each have the same meaning as m and n in the compound (2). Of those, the compound (3) is preferably a compound represented by the following formula (3′) where m+n is an integer of from 2 or more and 4 or less.

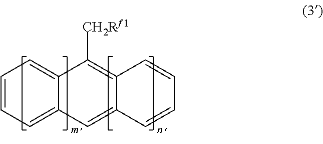

In the above formula, R^{f1} represents a fluorine-containing alkyl group having a carbon number of from 1 to 12, in which one or more fluorine atoms directly bond to the carbon atom that serves as an atomic bond, m′ is an integer of 1 or more, n′ is an integer of 0 or more, and m′+n′ is an integer of from 2 or more and 4 or less.

From the viewpoint of the solubility of the compound (3′) in organic solvents, m′ is more preferably 1 or 2, and n′ is more preferably 1 or 2.

As the compound (2) and the compound (3), preferred are the following compounds, respectively.

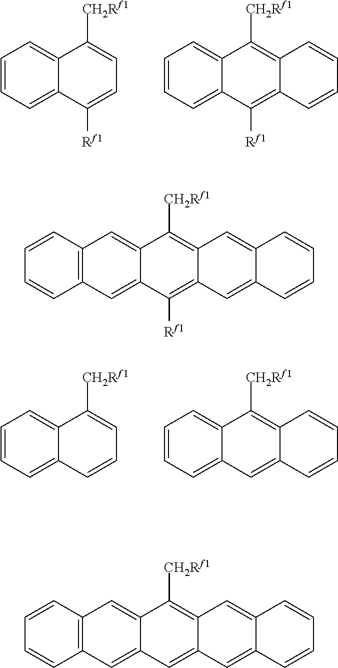

In the above formulae, R^{f1} represents a fluorine-containing alkyl group having a carbon number of from 1 to 12, in which one or more fluorine atoms directly bond to the carbon atom that serves as an atomic bond, preferably a perfluoroalkyl group having a carbon number of from 1 to 12, and especially preferably a perfluoroalkyl group having a carbon number of from 2 to 10. Preferred embodiments of these groups are the same as those mentioned above.

From the viewpoint of the solubility in organic solvents, R^{f1} preferably has a linear structure, and is therefore preferably a linear-structured perfluoroalkyl group having a carbon number of from 1 to 12, and more preferably a linear-structured perfluoroalkyl group having a carbon number of from 2 to 10.

### <Method for Producing Fluorine-Containing Compound>

The production method for the fluorine-containing compound represented by the formula (2) of the present invention is described below.

The present invention provides a method for producing a fluorine-containing compound represented by the following formula (2), which contains reacting a compound represented by the following formula (1) and a compound represented by a formula R^{f1}X (in which X represents iodine atom or bromine atom) under irradiation with light in a halogen-containing solvent and in the presence of a thiosulfate salt.

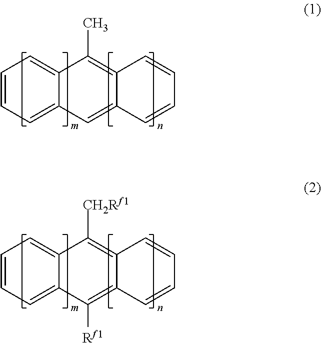

In the compound (2), R^{f1} represents a fluorine-containing alkyl group having a carbon number of from 1 to 12, in which one or more fluorine atoms directly bond to the carbon atom that serves as an atomic bond. In the formula (1) and the formula (2), m is an integer of 1 or more, n is an integer of 0 or more, and m+n is an integer of from 1 or more and 4 or less.

Exemplification and preferred embodiments of R^{f1} in the compound (2) as well as preferred embodiments of m and n therein are the same as those described in the section of describing the compound (2). Preferred embodiments of m and n in the compound (1) are the same as the preferred embodiments of m and n in the compound (2).

The present invention also provides a method for producing the fluorine-containing compound represented by the following formula (2) and a fluorine-containing compound represented by the following formula (3), which comprises reacting the compound represented by the following formula (1) and the compound represented by the formula R^{f1}X (in which X represents iodine atom or bromine atom) under irradiation with light in a halogen-containing solvent and in the presence of a thiosulfate salt. The compound (3) is the reaction intermediate in producing the compound (2).

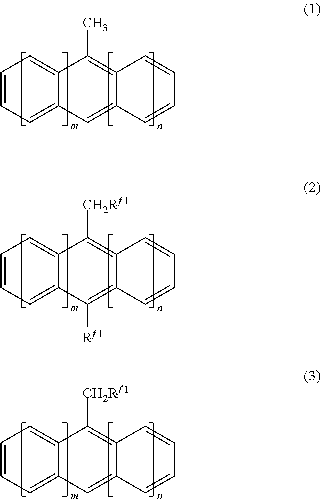

In the formulae (1) to (3), R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of from 1 or more and 4 or less.

In the compound (2) and the compound (3), R^{f1} is preferably a perfluoroalkyl group from the viewpoint of the fluorophilic effect. Concretely, preferred is a perfluoroalkyl group having a carbon number of from 1 to 12, and from the viewpoint of the balance between the intermolecular interaction and the improvement in solubility, more preferred is a perfluoroalkyl group having a carbon number of from 2 to 10.

From the viewpoint of the solubility in organic solvents, R^{f1} is preferably a linear-structured group, and is therefore more preferably a linear-structured perfluoroalkyl group having a carbon number of from 1 to 12, and even more preferably a linear-structured perfluoroalkyl group having a carbon number of from 2 to 10.

m and n in the compounds (1) to (3) have the same meanings as those of m and n mentioned in the section of describing the compound (2), respectively.

In the compounds (1) to (3), it is preferred that m is an integer of 1 or more, n is an integer of 1 or more and m+n is an integer of from 2 or more and 4 or less. From the viewpoint of the solubility in organic solvents, m is more preferably 1 or 2, and n is more preferably 1 or 2.

The halogen-containing solvent to be used in the synthesis is a solvent of an organic compound having a halogen atom. The halogen-containing solvent used in the present invention generally differs from the reactive substrate, R^{f1}X. Accordingly, the halogen-containing solvent is preferably a solvent containing a halogen atom except iodine atom and bromine atom, and the halogen atom in the solvent is preferably chlorine atom or fluorine atom. The halogen-containing solvent is preferably a halogenated aliphatic solvent. Above all, more preferred is a halogenated aliphatic hydrocarbon solvent or a halogenated ether solvent.

Examples of the halogen-containing solvent include chlorohydrocarbons, chlorofluorohydrocarbons, fluorine-containing ether compounds, etc. Concretely, usable here are methylene chloride, chloroform, 2,3,3-trichloroheptafluorobutane, 1,1,1,3-tetrachloro-2,2,3,3-tetrafluoropropane, 1,1,1-trichloropentafluoropropane, 1,1-dichloro-2,2,3,3,3,-pentafluropropane, 1,3-dichloro-1,2,2,3,3-pentafluoropropane, carbon tetrachloride, 1,2-dichloroethane, n-C₆F₁₃—C₂H₅, n-C₄F₉OCH₃, n-C₄F₉OC₂H₅, etc. Above all, preferred are chlorohydrocarbons such as methylene chloride, etc.; and chlorofluorohydrocarbons such as 1,1-dichloro-2,2,3,3,3-pentafluoropropane, 1,3-dichloro-1,2,2,3,3-pentafluoropropane, etc.; and more preferred is methylene chloride.

The amount of the halogen-containing solvent to be added is not specifically limited so long as it is only capable of dissolving the compound (1) as a starting material.

As the salt of the thiosulfate salt, any publicly-known or well-known compound is usable. Preferred is sodium thiosulfate or ammonium thiosulfate, and more preferred is sodium thiosulfate. The amount of the thiosulfate salt is preferably from 1 to 10 molar times and even more preferably from 3 to 6 molar times, relative to the compound (1).

In R^{f1}X, R^{f1} and X are the same as those mentioned in the section of describing the compound (2). From the viewpoint of the production yield, X is especially preferably iodine atom.

The amount of the compound represented by the formula R^{f1}X is preferably from 1 to 10 molar times, more preferably from 1 to 3 molar times, relative to the compound (1).

In the production step, water or the like may be added to the system in addition to the halogen-containing solvent, for securing the solubility of the thiosulfate salt. The amount of water is one capable of dissolving the thiosulfate salt, and is preferably from 2 to 100 g relative to 1 g of the thiosulfate salt.

As an example of the light source for photoirradiation that is employed in the reaction (photoreaction) under irradiation with light of the present invention, ultraviolet rays are mentioned. In a case where an ultraviolet ray is employed as the light source, in general, preferred is one capable of emitting an ultraviolet ray having a wavelength within a range of from 250 to 600 nm, which is used for chemical reaction, decomposition, sterilization, etc. Especially preferred is a high-pressure mercury lamp. The wavelength of the ultraviolet ray is preferably from 300 to 600 nm, more preferably from 330 to 470. For reaction under ultraviolet ray irradiation, any known photoirradiation apparatus is employable, and there may be mentioned a merry-go-round photoreaction apparatus, etc.

The photoirradiation time is preferably from 1 to 48 hours, and more preferably from 2 to 24 hours.

By using the above-mentioned photoirradiated reaction, R^{f1} can be introduced into the compound (1) to synthesize the compound (2) and the compound (3), without using any already-existing heavy metal coupling reaction. Another advantage is that the proportion of the heavy metal to be contained in the compound (2) or the compound (3) produced by this method is extremely small.

According to the production method of the present invention, the content of Ni, Cu, Zn and Pd to be contained in the compound (2) or the compound (3) can be at most 1 mass ppm each, and the total content of these heavy metals can be at most 10 mass ppm in the compound.

For organic semiconductor materials, heavy metal contamination may be one cause of carrier mobility degradation. Consequently, the heavy metal content is preferably as small as possible, and the organic semiconductor material using the compound (2) or the compound (3) obtained according to the production method of the present invention is expected to have excellent semiconductor characteristics.

The heavy metal content in the compound can be measured through atomic absorption analysis, etc.

The synthesis through photoradical reaction under photoirradiation as above can attain high position selectivity that could not be attained in thermochemical reaction. In the compound (2), the methyl group in the compound (1) as a starting material is first converted into a group —CH₂R^{f1}, and the group R^{f1} can be introduced into the para-position. The production method of the present invention enables introduction of —CH₂R^{f1} into an aromatic compound which, however, has heretofore been difficult.

The reaction product that contains the compound (2) or the compound (3) obtained from the compound (1) through the above-mentioned method can be separated and purified by means of any ordinary known method.

The solubility of the fluorine-containing compound of the present invention in these organic solvents is high, and above all, the compound exhibit extremely high solubility in hexane and cyclohexane that are known as low-polar solvents. Accordingly, the fluorine-containing compound can be easily purified by means of a simple method of column chromatography, recrystallization, etc.

### <Organic Semiconductor Material>

The organic semiconductor material is a material containing the fluorine-containing compound (2) of the present invention, and in use thereof, for example, the material may be mixed with any other organic semiconductor material, or may contain various dopants. For example, when the material is used as a light-emitting layer of an organic EL element, coumarin-type, quinacridone-type, rubrene-type, or stilbene-type derivatives, as well as fluorescent dyes and the like are usable as the dopant.

Owing to the affinity among the fluorine-containing alkyl groups, neighboring molecules may aggregate (fluorophilic effect), therefore contributing toward more efficient charge transfer. Consequently, using the fluorine-containing compound of the present invention can realize formation of organic semiconductor thin films having a high carrier mobility and can realize production of electronic elements such as transistors and the like utilizing the thin films.

In general, in the case where gold is used as an electrode material, an acene compound not having a substituent, such as anthracene, pentacene or the like acts as a p-type semiconductor. On the other hand, the fluorine-containing compound of the present invention has a fluorine-containing alkyl group that is an electron-attracting substituent, and therefore the electron transition energy thereof changes depending on the structure of the group. Accordingly, using the fluorine-containing compound of the present invention makes it possible to control the conductivity type of the organic semiconductor material.

### <Organic Semiconductor Thin Films>

The organic semiconductor material of the present invention can form an organic semiconductor film on a substrate through a dry-process or wet-process in accordance with an ordinary production method. The film includes a thin film, a thick film or a crystalline film.

In the case of forming a thin film through a dry process, a film can be formed by any known method such as a vacuum deposition method, an MBE (molecular beam epitaxy) method, a sputtering method, a laser vapor deposition method, a vapor-phase transportation growth method, etc.

These thin films and others can function as charge-transporting components of various functional elements such as photoelectric conversion elements, thin film transistor elements, light-emitting elements, etc., and therefore various electronic devices having such thin films, etc. can be produced.

In the case where a thin film is formed in accordance with a vacuum deposition method, an MBE method or a vapor-phase transportation growth method as a dry process, the organic semiconductor material is heated, and the sublimed vapor is transported onto the surface of a substrate in high vacuum, normal vacuum or low vacuum or under normal pressure. For the thin film formation, employable are any known method and condition. Concretely, it is preferable that the substrate temperature is from 20 to 200° C. and the thin film growth speed is from 0.001 to 1,000 nm/sec. Under the condition, a film exhibiting crystallinity and having a good surface smoothness in the thin film state can be formed.

When the substrate temperature is too low, the thin film would be readily amorphous, but when too high, the surface smoothness in the thin film state tends to worsen. When the thin film growth speed is too low, the crystallinity would readily lower, but when too high, the surface smoothness in the thin film state tends to worsen.

In the case where a wet process is employed, the organic semiconductor material containing the fluorine-containing compound of the present invention can be dissolved in an organic solvent to prepare a solution composition thereof, and this can be applied onto a substrate to coat it to form an organic semiconductor thin film thereon.

The fluorine-containing compound of the present invention is a compound which is improved in solubility in organic solvents as compared with already-existing organic semiconductor materials, and therefore has an advantage of applicability to a wet process. The reason is because, owing to the existence of the fluorine-containing alkyl group in the fluorine-containing compound, the organic semiconductor material of the present invention is lipophilic and is therefore soluble in various organic solvents. Consequently, the organic semiconductor material of the present invention is applicable to a wet process, and therefore can be processed without giving any damage to semiconductor materials.

The film formation method (substrate coating method) in a wet process includes application, spraying, contact, etc. Concretely, there are mentioned known methods such as a spin coating method, a casting method, a dip coating method, an inkjet method, a doctor blade method, a screen printing method, a dispenser method, etc. In the case of a tabular crystalline state or a thick film state, employable is a casting method or the like. Regarding the film formation method and the organic solvent, a combination thereof can be preferably selected to be suitable to the devices to be produced.

In a wet process, at least one selected from temperature gradient, electric field or magnetic field may be applied to the interface between the fluorine-containing compound solution and the substrate for controlling the crystal growth. By adopting this method, an organic semiconductor thin film having higher crystallinity can be produced, and excellent semiconductor characteristics based on the high-crystalline thin film performance can be achieved. By employing a solvent atmosphere as the ambient atmosphere during the film formation in a wet process, a high-crystalline organic semiconductor thin film can be produced by controlling the vapor pressure during solvent drying.

Examples of the organic solvent capable of dissolving the fluorine-containing compound (2) in a wet process include non-halogen organic solvents and halogen-containing organic solvents. The non-halogen organic solvents include aliphatic hydrocarbons such as pentane, hexane, heptane, etc.; alicyclic hydrocarbons such as cyclohexane, etc.; aromatic hydrocarbons such as benzene, toluene, xylene, phenol, cresol, etc.; ethers such as diethyl ether, tert-butyl methyl ether, tetrahydrofuran, dioxane, etc.; alcohols such as methanol, ethanol, 2-propanol, etc.; their mixtures, etc.

Examples of the halogen-containing organic solvents are as follows. For example, examples thereof include chlorohydrocarbons, fluorohydrocarbons, chlorofluorohydrocarbons, and fluorine-containing ether compounds. Concretely, employable here are methylene chloride, chloroform, 1,2-dichloroethane, chlorobenzene, 1,2-dichlorobenzene, 1,2,4-trichlorobenzene, 2,3,3-trichloroheptafluorobutane, 1,1,1,3-tetrachloro-2,2,3,3-tetrafluoropropane, 1,1,1-trichloro-2,2,3,3,3-pentafluoropropane, 1,1-dichloro-2,2,3,3,3-pentafluoropropane, 1,3-dichloro-1,1,2,2,3-pentafluoropropane, carbon tetrachloride, 1,2-dichloroethane, dichloropentafluoropropane, n-C₆F₁₃—C₂H₅, n-C₄F₉OCH₃, n-C₄F₉OC₂H₅, etc.

One of these solvents may be used singly or two or more thereof may be used as combined. In the case where two or more solvents are used as combined, preferably a non-halogen organic solvent and a halogen-containing organic solvent are combined, and preferably they are mixed in any ratio.

In the case where the fluorine-containing compound of the present invention is dissolved in an organic solvent to be used in a wet process, the amount of the organic semiconductor material in the organic solvent is preferably 0.01% by mass or more, and more preferably at least 0.2% by mass or so from the viewpoint of the process efficiency or the like. Further, the amount of the organic semiconductor material in the organic solvent is preferably from 0.01 to 10% by mass, and more preferably from 0.2 to 10% by mass.

Since the fluorine-containing compound of the present invention has excellent solubility in organic solvents, the fluorine-containing compound produced in the above-mentioned production method may be purified to have an increased purity by means of a simple purification method of column chromatography, recrystallization or the like.

Coating a substrate in a wet process may be carried out in air or in an inert gas atmosphere. In the case where the solution of the semiconductor material readily oxidizes, preferred is an inert gas atmosphere such as nitrogen, argon, etc.

After a substrate has been coated, the solvent is evaporated away to form an organic semiconductor thin film thereon. When the amount of the solvent remaining in the thin film is large, the stability and the semiconductor characteristics of the thin film may worsen. Therefore it is preferred that after the thin film formation, a follow-up heat treatment or a decompression treatment is conducted to remove the remaining solvent.

The shape of the substrate to be used in a wet process is not specifically limited. In general, a sheet-like or tabular substrate is used. The material used for the substrate includes ceramics, metal substrates, semiconductors, resins, papers, unwoven fabrics, etc.

The ceramics include substrates of glass, quartz, aluminium oxide, sapphire, silicon nitride, silicon carbide, etc. The metal substrates include substrates of gold, copper, silver, etc. The semiconductors include substrates of silicon (crystalline silicon, amorphous silicon), germanium, gallium arsenic, gallium phosphorus, gallium nitride, etc. The resins include substrates of polyesters, polyethylenes, polypropylenes, polyvinyls, polyvinyl alcohols, ethylene vinyl alcohol copolymers, cyclic polyolefins, polyimides, polyamides, polystyrenes, polycarbonates, polyether sulfones, polysulfones, polymethyl methacrylates, polyethylene terephthalates, triacetyl celluloses, norbornenes, etc.

The organic semiconductor thin film of the present invention is characterized in that the film is a crystalline thin film. Owing to a high crystallinity, the thin film can secure a high carrier mobility and can therefore express excellent organic semiconductor device characteristics.

The crystalline state of the thin film can be confirmed by a measurement of oblique-incidence X-ray diffractiometry or transmission electron diffractiometry of the thin film, or by a method of applying an X-ray to the edges of the thin film to analyze the diffraction thereof. Especially employed here is oblique-incidence X-ray diffractiometry that is a crystallographic method in the field of thin films. X-ray diffractiometry includes out-of-plane XRD method and in-plane XRD method that are differentiated depending on the direction of the lattice plane to be analyzed there. The out-of-plane XRD method is a method of analyzing the lattice plane parallel to the substrate, while the in-plane XRD method is a method of analyzing the lattice plane vertical to the substrate.

When it is said that the thin film is crystalline, it means that there can be observed diffraction peaks resulting from the organic semiconductor material that forms the thin film. Concretely, there are peaks resulting from the diffraction based on the crystal lattices of the organic semiconductor material, the diffraction derived from the molecular length, or the diffraction appearing characteristically when the molecules has an orientation in parallel or vertical to the substrate. Since when the thin film is in the amorphous state, these diffractions are not observed, the thin film that has given such diffraction peaks can be said to be a crystalline thin film.

The thickness of the organic semiconductor thin film layer for use in organic semiconductor elements is preferably from 10 to 1,000 nm.

### <Organic Semiconductor Element, Organic Semiconductor Transistor>

The fluorine-containing compound of the present invention has a high carrier mobility. Accordingly, the organic semiconductor material containing the compound can form an organic semiconductor thin film without detracting from the high carrier mobility of the compound.

The organic semiconductor element that contains a layer of the organic semiconductor thin film as a semiconductor layer therein is extremely useful for various semiconductor devices.

In the organic semiconductor thin film, the long axis of the molecule of the fluorine-containing compound of the present invention is preferably aligned in the vertical direction relative to the surface of the substrate.

Examples of the semiconductor device include organic semiconductor transistors, organic semiconductor lasers, organic photoelectric conversion devices, organic molecular memories, etc. Above all, preferred are organic semiconductor transistors, and more preferred are organic field effect transistors (organic FETs).

The organic semiconductor transistor generally contains a substrate, a gate electrode, an insulation layer (dielectric layer), a source electrode, a drain electrode, and a semiconductor layer. In addition, it may contain a back gate, a bulk, etc.

The sequence of arrangement of the constitutive components in the organic semiconductor transistor is not specifically limited. Of the above-mentioned constitutive components, the gate electrode, source electrode, drain electrode, and semiconductor layer each may be present as plural layers. In the case where plural semiconductor layers are present, the layers may be arranged on one plane or may be laminated.

### EXAMPLES

The present invention is described concretely with reference to the Examples as follows, however, the present invention is not whatsoever restricted by these Examples.

### (Evaluation Method)

In the Examples, the structure of the synthesized compound was identified under the following condition by using a Fourier transform high-resolution nuclear magnetic resonance device (NMR, JNM-AL400, by JEOL). In NMR, the multiplicity was abbreviated as follows, singlet: s, doublet: d, triplet: t, quartet: q, multiplet: m, and broad: br. The details of the measurement condition are shown below.

### (NMR)

¹H NMR (400 MHz) solvent: chloroform-d (CDCl₃), internal standard: tetramethylsilane (TMS).

¹⁹F NMR (376 MHz): solvent: chloroform-d (CDCl₃).

¹³C NMR (125 MHz): solvent: chloroform-d (CDCl₃), internal standard: chloroform-d (CDCl₃).

For the mass spectrometry, used was Extractive manufactured by Thermofisher or JMF-S3000 Spiral TOF (MALDI-TOFMS) manufactured by JEOL. In Extractive, the sample was dissolved in methanol, and then measured by using ESI or APCI as the ionization method. In MALDI-TOFMS, the sample was dissolved in tetrahydrofuran to be 0.2% by mass, then mixed with a cationizing agent, and analyzed. As the cationizing agent, used was a 0.1 mass % sodium iodide/acetonitrile solution.

In the Examples, the progress status of the series of synthesis reaction was appropriately confirmed through thin layer chromatography (TLC, silica gel 60 F254, manufactured by Merck).

As the ultraviolet ray source for photoreaction, used was an ultraviolet ray lamp (UVG-11, manufactured by Ultraviolet).

For solvent removal, used was a rotary evaporator in every case.

In the column chromatography, silica gel was used as the columns, and the amount of each fraction was made to correspond to 1 mL per 1 g of the silica gel used. The necessary fraction was collected, then the solvent was evaporated away by using a rotary evaporator, and the residue was dried under reduced pressure. As the silica, used was silica gel 60 FC (spherical) (manufactured by Kanto Chemical). As the developing solvent, used was hexane (manufactured by Godo).

### Example 1-1

### Synthesis of Compound (a-1) Perfluorohexylated Anthracene

In a Pyrex® tube, 9-methylanthracene (manufactured by Tokyo Chemical, 38.4 mg, 0.2 mmol) was dissolved in methylene chloride (manufactured by Kanto Chemical, 5 mL), and thereto were further added nC₆F₁₃I (manufactured by Daikin, 86.7 μL, 0.4 mmol), sodium thiosulfate (manufactured by Kanto Chemical, 0.1581 g, 1 mmol) and water (1 mL), followed by irradiating with ultraviolet rays for 6 hours by using a 450 W high-pressure mercury lamp while the temperature of the reaction system was kept constant by applying cold running water thereto. After irradiation with ultraviolet rays, the aqueous layer was removed, the reaction solution was extracted with methylene chloride, and then the organic layer was dried with anhydrous sodium sulfate and filtered. The filtrate was concentrated, and separated and purified through column chromatography to give a compound represented by the following formula (a-1) as a white solid (0.1424 g, yield 86%).

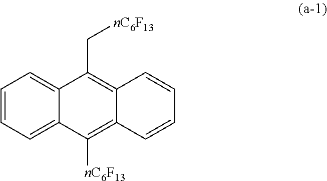

The analysis results in NMR of the resultant compound (a-1) are shown below.

¹H NMR: δ 8.46 to 8.44 (2H, m, Ph), 8.24 to 8.22 (2H, m, Ph), 7.62 to 7.60 (4H, m, Ph), 4.53 (2H, t, J=18 Hz, CH₂).

¹⁹F NMR: δ-80.7 (6F, s, 2CF₃), −91.4 (2F, s, CF₂), −109.7 (2F₁₃ s, CF₂), −117.9 (2F, s, CF₂), −121.4 (4F, s, 2CF₂), −122.4 to −122.8 (6F, m, 3CF₂), −125.9 (4F, s, 2CF₂).

¹³C NMR: δ 131.7(s), 131.1(s), 128.8(s), 127.1(s), 126.3(s), 126.1 to 125.8(m), 124.8(s), 29.1 (t, J=26 Hz, CH₂).

The result of MALDI-TOFMS of the resultant compound (a-1) was: C₂₇H₁₀F₂₆ [M+], calculated value 828.0362, and found value 828.0369.

### Example 1-2

### Synthesis of Compound (a-2) Perfluorohexylated Anthracene

In the synthesis process for the above compound (a-1), the product in the step prior to filtration of the reaction solution that had been extracted with methylene chloride after the photoreaction was mass-analyzed, thereby confirming the formation of a compound represented by the following formula (a-2). The result of MALDI-TOFMS was: C₂₁H₁₁F₁₃ [M+], calculated value 510.0653 and found value 510.0662.

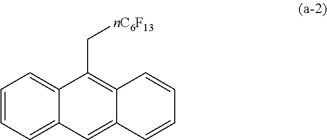

### Example 2-1

### Synthesis of Compound (b-1) Perfluorohexylated Naphthalene

In a Pyrex® tube, 1-methylnaphthalene (manufactured by Wako Pure Chemical Industries, 71.1 mg, 0.5 mmol) was dissolved in methylene chloride (manufactured by Kanto Chemical, 12.5 mL), and thereto were further added nC₆F₁₃I (manufactured by Daikin, 0.22 mL, 1.0 mmol), sodium thiosulfate (manufactured by Kanto Chemical, 0.3716 g, 2.5 mmol) and water (2.5 mL), followed by irradiating with ultraviolet rays for 24 hours by using a 450 W high-pressure mercury lamp while the temperature of the reaction system was kept constant by applying cold running water thereto. After irradiation with ultraviolet rays, the aqueous layer was removed, the reaction solution was extracted with methylene chloride, and then the organic layer was dried with anhydrous sodium sulfate and filtered. The filtrate was concentrated, and separated and purified through column chromatography to give a compound represented by the following formula (b-1) as a white solid (0.2140 g, yield 55%).

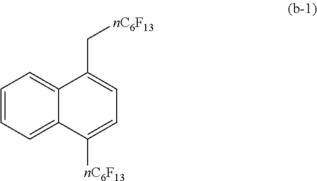

The analysis results in NMR of the resultant compound (b-1) are shown below.

¹H NMR: δ 8.31 to 8.29 (1H, m, Ph), 8.09 to 8.10 (1H, m, Ph), 7.84 and 7.82 (1H, d, J=7.6 Hz, Ph), 7.68 to 7.65 (2H, m, Ph), 7.59 and 7.57 (1H, d, J=8.0 Hz, Ph), 3.92 (2H, t, J=19 Hz, CH₂).

¹⁹F NMR: δ−80.7 (6F, s, 2CF₃), −104.1 (2F, s, CF₂), −111.6 (2F, s, CF₂), −119.9 (2F, s, CF₂), −121.3 to −121.5 (6F, m, 3CF₂), −122.7 (4F, s, 2CF₂), −125.9 (4F, s, 2CF₂).

¹³C NMR: δ 116.9(s), 114.4(s), 114.2(s), 112.3(s), 111.1(s), 110.8(s), 110.7(s), 110.6(s), 109.1 to 109.0(m), 107.9(s), 17.2 (t, J=23 Hz, CH₂).

The result of MALDI-TOFMS of the resultant compound (b-1) was: C₂₃H₈F₂₆ [M+], calculated value 778.0205, and found value 778.0193.

### Example 2-2

### Synthesis of Compound (b-2) Perfluorohexylated Naphthalene

In the synthesis process for the above compound (b-1), the product in the step prior to filtration of the reaction solution that had been extracted with methylene chloride after the photoreaction was mass-analyzed, thereby confirming the formation of a compound represented by the following formula (b-2). The result of MALDI-TOFMS was: C₁₇H₉F₁₃[M+], calculated value 460.0497 and found value 460.0488.

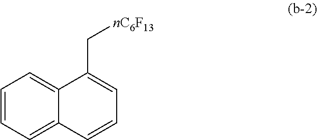

Further, compounds corresponding to the compound (a-1) where the nC₆F₁₃ moiety is changed to a trifluoromethyl group, a perfluoroethyl group, a perfluoro-n-propyl group, a perfluoro-n-butyl group, a perfluoro-n-heptyl group or a perfluoro-n-octyl group can also be produced by using the corresponding starting materials and by performing similar reaction as above.

### Comparative Example 1

In a Pyrex® tube, 9,10-dimethylanthracene (manufactured by Wako Pure Chemical Industries, 41.2 mg, 0.2 mmol) was dissolved in methylene chloride (manufactured by Kanto Chemical, 5 mL), and thereto were further added nC₆F₁₃I (manufactured by Daikin, 86.6 μL, 0.4 mmol), sodium thiosulfate (manufactured by Kanto Chemical, 0.1581 g, 1 mmol) and water (1 mL), followed by irradiating with ultraviolet rays for 12 hours by using a 450 W high-pressure mercury lamp while the temperature of the reaction system was kept constant by applying cold running water thereto. After irradiation with ultraviolet rays, the aqueous layer was removed, and the reaction solution was extracted with methylene chloride. The product was mass-analyzed, but the result indicated that a compound represented by the following formula (c) could not be produced. In other words, though the reaction went on, but the intended compound could not be obtained and it is considered that decomposition or the like would have gone on.

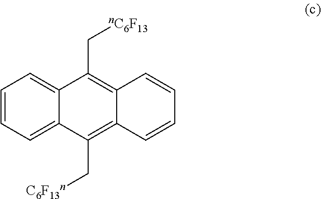

### Comparative Example 2

In a Pyrex® tube, 1-propylnaphthalene (manufactured by Wako Pure Chemical Industries, 85.1 mg, 0.5 mmol) was dissolved in methylene chloride (manufactured by Kanto Chemical, 12.5 mL), and thereto were further added nC₆F₁₃I (manufactured by Daikin, 0.22 mL, 1.0 mmol), sodium thiosulfate (manufactured by Kanto Chemical, 0.3716 g, 2.5 mmol) and water (2.5 mL), followed by irradiating with ultraviolet rays for 12 hours by using a 450 W high-pressure mercury lamp while the temperature of the reaction system was kept constant by applying cold running water thereto. After irradiation with ultraviolet rays, the aqueous layer was removed, and the reaction solution was extracted with methylene chloride. The product was mass-analyzed, and the result indicated that a compound represented by the following formula (d) could not be produced, but a compound represented by the following formula (e) was produced.

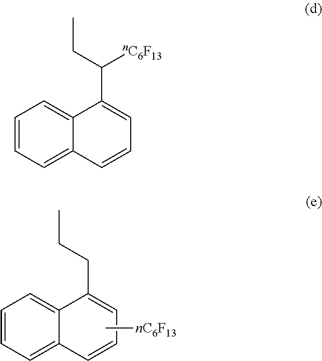

### <Solubility Test>

For evaluating the applicability thereof to a wet process, the compound to be analyzed was tested in a dissolution test in various solvents. As a comparative case, anthracene was tested for the dissolution test.

Concretely, 20 mg of the sample was weighed, and the solubility thereof (0.2 mass %) in 10 g of a solvent at room temperature was visually evaluated.

The type of the solvents and the result are shown in Table 1 below. In Table 1, A means soluble, and B means insoluble. The standard of “soluble” was set as the case where the compound dissolved in an amount of 0.2% by mass or more at a solvent temperature of 25° C.

TABLE 1

| Solubility Test | Hexane | Cyclohexane |
|---|---|---|
| Example 1-1 | A | A |
| Compound (a-1) | | |
| Comparative Example 3 | B | B |
| Anthracene | | |

From the above results, it becomes obvious that the fluorine-containing compound of the present invention has a high solubility in organic solvents, as compared with anthracene. This may be considered because a perfluoroalkyl group was introduced into the compound.

As a result, it may be said that the compound is applicable to a wet process.

### <Organic Semiconductor Material Characteristics>

For characteristics evaluation of the compound (a-1) as an organic semiconductor material, a vapor deposition field effect transistor (vapor-deposition FET) element was produced and the electric field effect mobility (carrier mobility) thereof was determined. The production method for the vapor-deposition FET element and the semiconductor characteristics evaluation method are described below.

A washed, silicon oxide film-attached silicon substrate was immersed in a toluene solution of n-octyltrichlorosilane to thereby process the surface of the silicon oxide film. On the substrate, the compound (a-1) obtained in Example 1-1 was vacuum-vapor-deposited (back pressure: about 10⁻⁴ Pa, vapor deposition rate 0.1 angstrom/s, substrate temperature 25° C., film thickness: 100 nm), thereby forming an organic semiconductor layer.

Via a shadow mask, gold was vacuum-vapor-deposited on the top of the organic semiconductor layer (back pressure: about 10⁻³ Pa, vapor deposition rate 1 to 2 angstrom/s, film thickness: 50 nm), thereby forming source and drain electrodes (channel length 50 μm, channel width 1 mm). The organic semiconductor layer and the silicon oxide film in the area differing from the electrode were cut away, an electroconductive paste (manufactured by Fujikura Chemical, Dotite D-550) was stuck to that area, and the solvent was dried away. In that manner, a top contact/bottom gate structured electric field effect transistor (FET) element was produced.

The electric characteristics of the resultant vapor-deposited FET element were evaluated in vacuo (<5×10⁻³ Pa) by using a semiconductor device analyzer B1500A manufactured by Agilent. The silicon substrate of the produced, vapor-deposited FET element was made to serve as a gate electrode, a voltage was applied to the silicon substrate, and a current/voltage curve between the source/drain electrodes was determined by scanning the gate voltage.

As a result, the on/off motion of the drain current due to the gate voltage of the vapor-deposited FET element was observed, and from the inclination of the drain current/gate voltage, the electric field effect mobility (carrier mobility) was determined. The organic semiconductor element produced by using the compound (a-1) showed characteristics of a p-type transistor element.

From the saturation region of the current/voltage characteristic of the organic thin film transistor, the carrier mobility was calculated, and was 1.9×10⁻⁶ cm²/V·s in vacuo.

While the present invention has been described in detail and with reference to specific embodiments thereof, it will be apparent to one skilled in the art that various changes and modifications can be made therein without departing from the spirit and scope thereof. The present application is based on a Japanese patent application (Patent Application No. 2012-280153) filed on Dec. 21, 2012, and the contents thereof are herein incorporated by reference.

### INDUSTRIAL APPLICABILITY

The present invention provides a novel fluorine-containing compound usable in both a dry process and a wet process and being expected to have a high mobility, and provides a method for producing the compound.

In the fluorine-containing compound, a fluorine-containing alkyl group can be introduced without using metal coupling reaction, and therefore, the organic semiconductor material containing the fluorine-containing compound can dissolve in a low-polar solvent and can evade the risk of heavy metal contamination, and such a fluorine-containing compound having a high carrier mobility can be obtained.

Owing to the high carrier mobility through π-π stacking in aromatic moieties and owing to the fluorophilic effect of the fluorine-containing alkyl group, the organic semiconductor material containing the compound is useful as a material usable for organic thin film transistors, organic EL elements for next-generation flat panel displays, organic thin film solar cells as lightweight and flexible power sources, etc.

1. A method comprising:
reacting a compound represented by the following formula (1) and a compound represented by a formula R^{f1}X under light irradiation in a halogen-containing solvent in the presence of a thiosulfate salt; and
thereby obtaining a fluorine-containing compound represented by the following formula (2) and/or a fluorine-containing compound represented by the following formula (3),
wherein X represents an iodine atom or a bromine atom,
R^{f1} represents a fluorine-containing alkyl group having a carbon number of from 1 to 12, in which one or more fluorine atoms directly bond to the carbon atom that serves as an atomic bond,
m is an integer of 1 or more, n is an integer of 0 or more, and m+n is an integer of 1 or more and 4 or less;

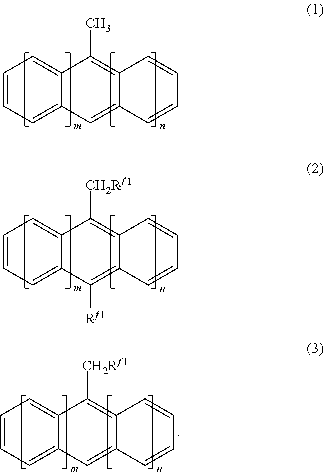

2. The method according to claim 1, wherein R^{f1} is a perfluoroalkyl group having a carbon number of from 1 to 12.

3. The method according to claim 2, wherein R^{f1} is a linear-structured perfluoroalkyl group having a carbon number of from 1 to 12, m is an integer of 1 or more, n is an integer of 1 or more, and m+n is an integer of 2 or more and 4 or less.

4. The method according to claim 1, obtaining the fluorine-containing compound represented by the formula (2).

5. The method according to claim 4, wherein R^{f1} is a perfluoroalkyl group having a carbon number of from 1 to 12.

6. The method according to claim 5, wherein R^{f1} is a linear-structured perfluoroalkyl group having a carbon number of from 1 to 12, m is an integer of 1 or more, n is an integer of 1 or more, and m+n is an integer of 2 or more and 4 or less.

7. The method according to claim 1, obtaining the fluorine-containing compound represented by the formula (3).

8. The method according to claim 7, wherein R^{f1} is a perfluoroalkyl group having a carbon number of from 1 to 12.

9. The method according to claim 8, wherein R^{f1} is a linear-structured perfluoroalkyl group having a carbon number of from 1 to 12, m is an integer of 1 or more, n is an integer of 1 or more, and m+n is an integer of 2 or more and 4 or less.

10. A fluorine-containing compound represented by the following formula (2),
wherein R^{f1} represents a fluorine-containing alkyl group having a carbon number of from 1 to 12, in which one or more fluorine atoms directly bond to the carbon atom that serves as an atomic bond, m is an integer of 1 or more, n is an integer of 0 or more, and m+n is an integer of 1 or more and 4 or less:

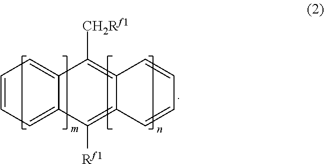

11. The fluorine-containing compound according to claim 10, wherein R^{f1} is a perfluoroalkyl group having a carbon number of from 1 to 12.

12. The fluorine-containing compound according to claim 11, wherein R^{f1} is a linear-structured perfluoroalkyl group having a carbon number of from 1 to 12, m is an integer of 1 or more, n is an integer of 1 or more, and m+n is an integer of 2 or more and 4 or less.

13. A compound represented by the following formula (3′)
wherein R^{f1} represents a fluorine-containing alkyl group having a carbon number of from 1 to 12, in which one or more fluorine atoms directly bond to the carbon atom that serves as an atomic bond, m′ is an integer of 1 or more, n′ is an integer of 0 or more, and m′+n′ is an integer of 2 or more and 4 or less; [chemical structure]
